(19) United States
(12) Patent Application Publication
(10) Pub. No.: US 2021/0169549 A1
(43) Pub. Date: Jun. 10, 2021

(54) PHOTODYNAMIC THERAPY ABLATION DEVICE

(71) Applicant: Acclarent, Inc., Irvine, CA (US)

(72) Inventors: Jetmir Palushi, Irvine, CA (US); Athanasios Papadakis, Newport Beach, CA (US); Itzhak Fang, Irvine, CA (US); Henry F. Salazar, Pico Rivera, CA (US); Jordan R. Trott, Redondo Beach, CA (US); Raymond L. Weiss, JR., Ocean Springs, MS (US)

(21) Appl. No.: 17/095,980

(22) Filed: Nov. 12, 2020

Related U.S. Application Data
(60) Provisional application No. 62/943,287, filed on Dec. 4, 2019.

(51) Int. Cl. A61B 18/06; A61N 5/06; A61B 18/08; A61L 29/08; A61L 29/16

(52) U.S. Cl. CPC A61B 18/06; A61N 5/062; A61B 2018/00327; A61L 29/085; A61L 29/16; A61B 18/082

(57) ABSTRACT

A method of ablating tissue of a target anatomical structure in an ear, a nose, or a throat of a patient includes inserting a balloon catheter into the ear, the nose, or the throat. The distal end of the balloon catheter is advanced distally until the balloon is disposed in the target anatomical structure. The balloon is inflated to an expanded configuration in the target anatomical structure. The tissue is ablated using at least one of photodynamic therapy or heated inflation fluid within the balloon. For photodynamic therapy, a photosensitizer is exposed to a light to produce a form of oxygen to ablate the target anatomical structure when the balloon is in the expanded configuration. For heated inflation fluid, heat is applied by a heating element to heat the inflation fluid in the balloon and thereby ablate the target anatomical structure when the balloon is in the expanded configuration.

### PRIORITY

This application claims priority to U.S. Provisional Patent Application Ser. No. 62/943,287, filed Dec. 4, 2019, entitled “Photodynamic Therapy Ablation Device,” the disclosure of which is incorporated by reference herein.

### BACKGROUND

To open a blocked target anatomical structure or otherwise treat a targeted anatomical structure, it may be beneficial to expand and/or ablate the target anatomical structure using a dilation catheter that includes an inflatable balloon. One such balloon catheter that is capable of expanding and/or ablating the target anatomical structure is shown and described in U.S. Pat. No. 10,485,609, entitled “Dilation Balloon with RF Energy Delivery Feature,” issued on Nov. 26, 2019, the disclosure of which is incorporated by reference herein.

In some cases, mucosa thickening within the target anatomical structure (e.g. a sinus cavity, a Eustachian tube (ET), or another passageway) after balloon dilation may cause multiple complications including a blockage of the opened target anatomical structure. Ablation may help correct the mucosa thickening within the target anatomical structure, but ablation may cause possible charring of the tissue. As a result, it may be desirable to more gently ablate the target anatomical structure without any charring of the tissue to better trigger healthy cell regeneration (similar to the ET). Additionally, it may be desirable to more globally ablate the target anatomical passageway in the patient in a straightforward and cost-effective manner. For example, it may be beneficial to ablate the ostia of paranasal sinuses, the larynx, the Eustachian tube, or other passageways within the ear, nose, or throat.

While several systems and methods have been made and used to ablate anatomical cavities, it is believed that no one prior to the inventors has made or used the invention described in the appended claims.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements and in which:

FIG. 1 depicts a front view of a distal end of an exemplary guide being advanced to an anatomical structure within a nose of a patient;

FIG. 2 depicts a schematic side elevational view of a kit that includes the guide positioned within the target anatomical structure of FIG. 1, with an exemplary balloon catheter being advanced distally through a lumen of the guide;

FIG. 2A depicts an enlarged sectional side view of the kit that includes the guide and the balloon catheter of FIG. 2;

FIG. 2B depicts a cross-sectional view of the kit of FIG. 2A taken along line 2B-2B of FIG. 2A;

FIG. 3 depicts a schematic side elevational view of the distal end of the balloon catheter of FIG. 2, but with the balloon positioned in the target anatomical structure in a contracted configuration;

FIG. 3A depicts an enlarged sectional side view of the balloon catheter within the target anatomical structure of FIG. 3;

FIG. 3B depicts a cross-sectional view of the balloon catheter within the target anatomical structure of FIG. 3A taken along line 3B-3B of FIG. 3A;

FIG. 4 depicts a schematic side elevational view of the distal end of the balloon catheter positioned within the target anatomical structure of FIG. 3, but with a pressure source expanding the balloon to an expanded configuration;

FIG. 4A depicts an enlarged schematic sectional side view of the balloon catheter within the target anatomical structure of FIG. 4;

FIG. 4B depicts a cross-sectional view of the balloon catheter within the target anatomical structure of FIG. 4A taken along line 4B-4B of FIG. 4A;

FIG. 5 depicts a schematic side elevational view of the distal end of the balloon catheter positioned within the target anatomical structure of FIG. 4, but with an ablation feature providing light or heat to ablate the target anatomical structure;

FIG. 5A depicts an enlarged schematic sectional side view of the balloon catheter within the target anatomical structure of FIG. 5;

FIG. 5B depicts a cross-sectional view of the balloon catheter within the target anatomical structure of FIG. 5A taken along line 5B-5B of FIG. 5A;

FIG. 6 depicts a schematic side elevational view of the distal end of the balloon catheter positioned within the target anatomical structure of FIG. 5, but with the pressure source contracting the balloon assembly to a contracted configuration;

FIG. 6A depicts an enlarged schematic sectional side view of the balloon catheter within the target anatomical structure of FIG. 6;

FIG. 6B depicts a cross-sectional view of the balloon catheter within the target anatomical structure of FIG. 6A taken along line 6B-6B of FIG. 6A;

FIG. 7 depicts a schematic side elevational view of the distal end of the balloon catheter of FIG. 6 and the guide of FIG. 1, but with the balloon catheter removed from the target anatomical structure using the guide;

FIG. 7A depicts an enlarged schematic sectional side view of the balloon catheter and the guide of FIG. 7;

FIG. 7B depicts a cross-sectional view of the balloon catheter and the guide of FIG. 7A taken along line 7B-7B of FIG. 7A;

FIG. 8 depicts a schematic perspective view of the balloon catheter of FIG. 5 ablating the target anatomical structure but with the light positioned outside of the balloon;

FIG. 9 depicts a schematic perspective view of the balloon catheter of FIG. 5 ablating the target anatomical structure but with the heating element positioned outside of the balloon; and

FIG. 10 depicts a diagrammatic view of the exemplary method.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

It will be appreciated that the terms “proximal” and “distal” are used herein with reference to a clinician gripping a handpiece assembly. Thus, an end effector is distal with respect to the more proximal handpiece assembly. It will be further appreciated that, for convenience and clarity, spatial terms such as “top” and “bottom” also are used herein with respect to the clinician gripping the handpiece assembly. However, surgical instruments are used in many orientations and positions, and these terms are not intended to be limiting and absolute.

It is further understood that any one or more of the teachings, expressions, versions, examples, etc. described herein may be combined with any one or more of the other teachings, expressions, versions, examples, etc. that are described herein. The following-described teachings, expressions, versions, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

### I. Exemplary Method for Ablation of Target Anatomic Structure

A. Exemplary Kit With Exemplary Guide and Exemplary Balloon Catheter

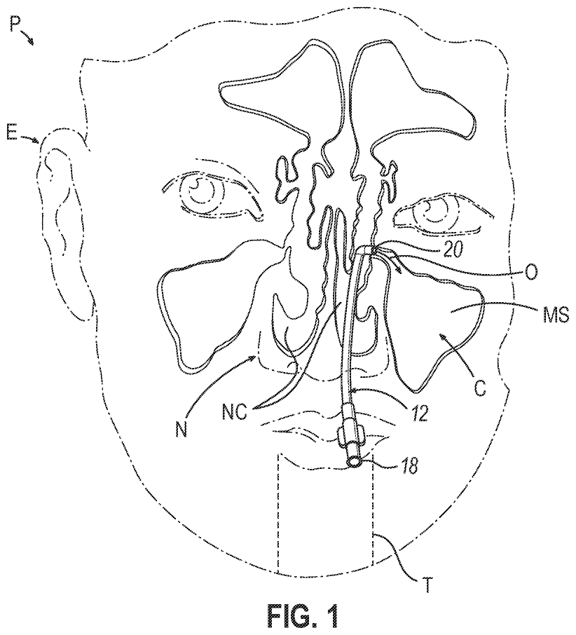

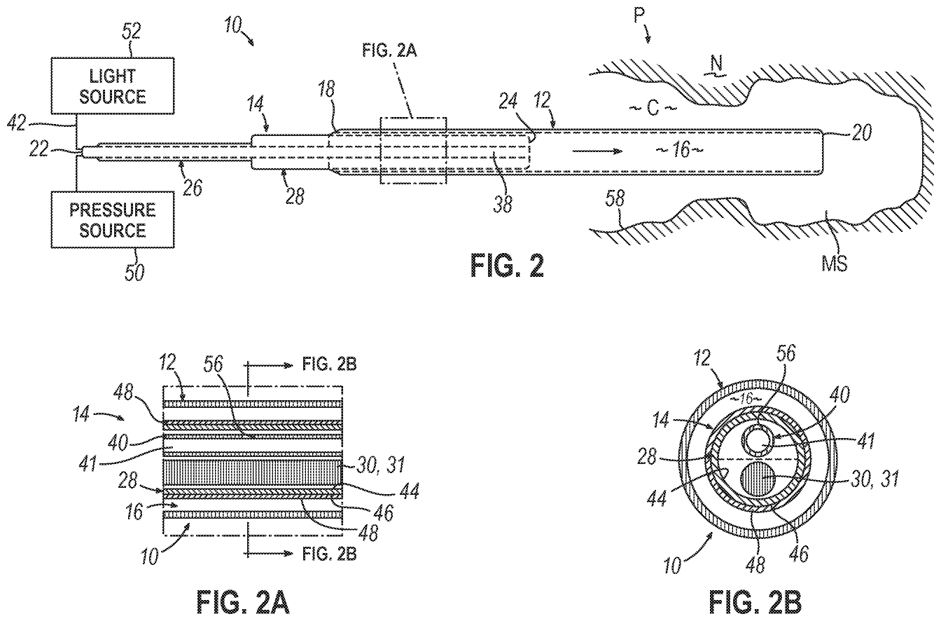

A method of ablating a target anatomical structure in an ear (E), a nose (N), or a throat (T) of a patient (P) is shown and described with reference to the following Figures. The method may be performed using an exemplary kit (10) that includes an exemplary guide (12) and an exemplary balloon catheter (14) (see FIG. 2). Particularly, FIG. 1 shows a front view of a distal end (20) of guide (12) being inserted and positioned within a target anatomical structure (C) prior to insertion of balloon catheter (14). Guide (12) includes a lumen (16) extending between proximal and distal ends (18, 20). Lumen (16) is sized and configured for balloon catheter (14) to slidably extend therein. By way of example only, some other variations may provide introduction of balloon catheter (14) via another instrument (e.g. a guide catheter or endoscope (not shown)). However, the use of guide (12) is optional, such that balloon catheter (14) may be advanced to the desired position (e.g. the target anatomical structure (C)), without the use of guide (12) or another instrument to assist in such introduction.

As shown in FIG. 1, distal end (20) of guide (12) is inserted into the nose (N) of the patient (P) patient and routed through a nasal cavity (NC). Guide (12) may be straight or have a bent distal portion. The bend may be rigid, malleable, or actively steerable (e.g. via one or more pull wires (not shown)). In the procedure of the present example, guide (12) may be inserted transnasally and advanced through the nasal cavity (NC) through a sinus ostium (O) to a position within or near the target anatomical structure (C) to be ablated. As used herein, “target anatomical structure” is intended to describe any close-ended crevice or open-ended passageway within the ear (E), the nose (N), or the throat (T) of patient (P). While in the present example, the target anatomical structure (C) is shown and described as the maxillary sinus (MS) accessed through the ostium (O), a variety of other suitable target anatomical cavities of the ear (E), the nose (N), or the throat (T) are also envisioned. As such, the target anatomical structure (C) may vary from the shown target anatomical structure (C). By way of example only, the target anatomical structure (C) may correspond to a Eustachian tube, a larynx, a choana, a sphenoid sinus ostium, one or more openings associated with one or more ethmoid sinus air cells, the frontal recess, and/or other passageways associated with paranasal sinuses. Additionally, while the shown target anatomical structure (C) is hourglass shaped, it is envisioned that the target anatomical structure (C) may have a variety of shapes and sizes that are suitable for use with kit (10) or balloon catheter (14).

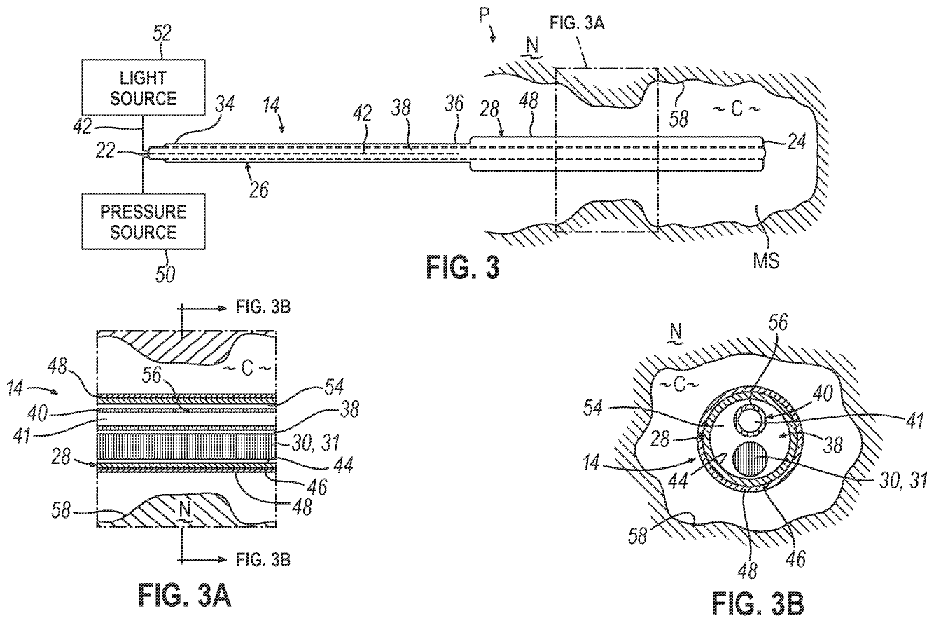

As shown in FIG. 2-2B, after guide (12) has been positioned, the operator may distally advance balloon catheter (14) through lumen (16) of guide (12). As shown, balloon catheter (14) includes proximal and distal ends (22, 24). Particularly, FIG. 2 shows a schematic side elevational view of kit (10) that includes guide (12) positioned within the target anatomical structure (C). FIG. 2A shows an enlarged sectional view of kit (10) of FIG. 2. FIG. 2B shows a cross-sectional view of kit (10) of FIG. 2A taken along line 2B-2B of FIG. 2A. As shown in FIGS. 3-6A, the upwardly and downwardly sloping lines define the target anatomical structure (C), where the target anatomical structure (C) is the space therebetween. Guide (12), described above as optional, may be removed once balloon (28) is disposed in the target anatomical structure (C). Alternatively, guide (12) may remain in place, with balloon (28) being positioned distally of the distal end of guide (12).

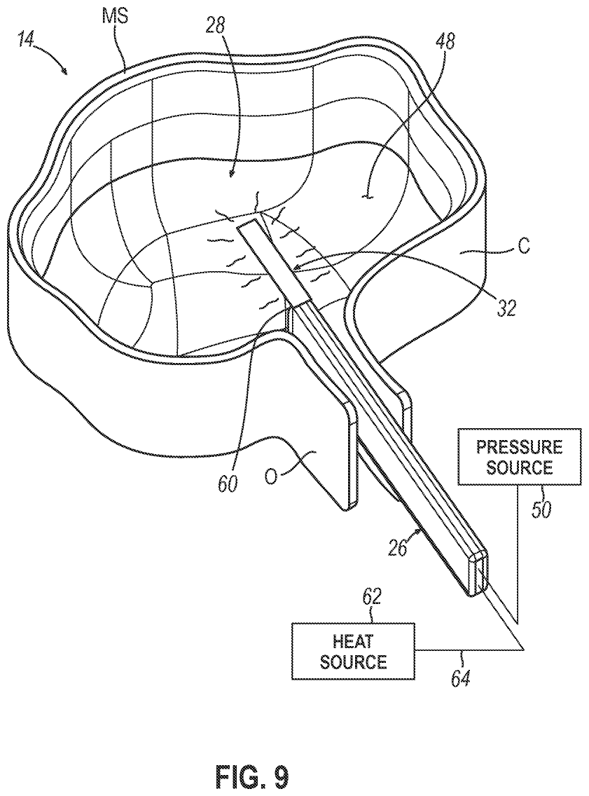

Balloon catheter (14) includes a shaft (26), a balloon (28), and an ablation feature (30). Ablation feature (30) may include at least one light emitting diode (31)(see FIGS. 2A-8) or at least one heating element (32) (see FIG. 9) as described below. While FIGS. 2A-7B show ablation feature (30) as including LED (31), it is also envisioned that ablation feature (30) may include heating element (32) (as described below with reference to FIG. 9) instead of, or in addition to, LED (31). Ablation feature (30) (e.g. LED (31) or heating element (32)) may be operatively coupled with distal portion (36) of shaft (26) defining a longitudinal axis (LA). Ablation feature (30) is configured to ablate target anatomical structure (C) of ear (E), the nose (N), or the throat (T) of patient (P) using photodynamic therapy or using heated inflation fluid disposed within interior (54) of balloon (28). Ablation feature (30) may be fixably coupled with balloon catheter (14). As shown, LED (31) is fixably coupled to distal portion (36) of shaft (26) of balloon catheter (14).

Balloon catheter (14) is sized and configured to ablate the target anatomical structure (C) in the ear (E), the nose (N), or the throat (T). Shaft (26) includes proximal and distal portions (34, 36) and a lumen (38) extending therethrough. An inflation tube (40) and a wire (42) may extend through lumen (38) as described in greater detail below. Balloon (28) is coupled with distal portion (36) of shaft (26). For example, balloon (28) may be permanently coupled with distal portion (36) of shaft (26) using a variety of coupling methods (e.g. adhesive bonding). In other words, balloon (28) may be fixably secured to distal portion (36) of shaft (26).

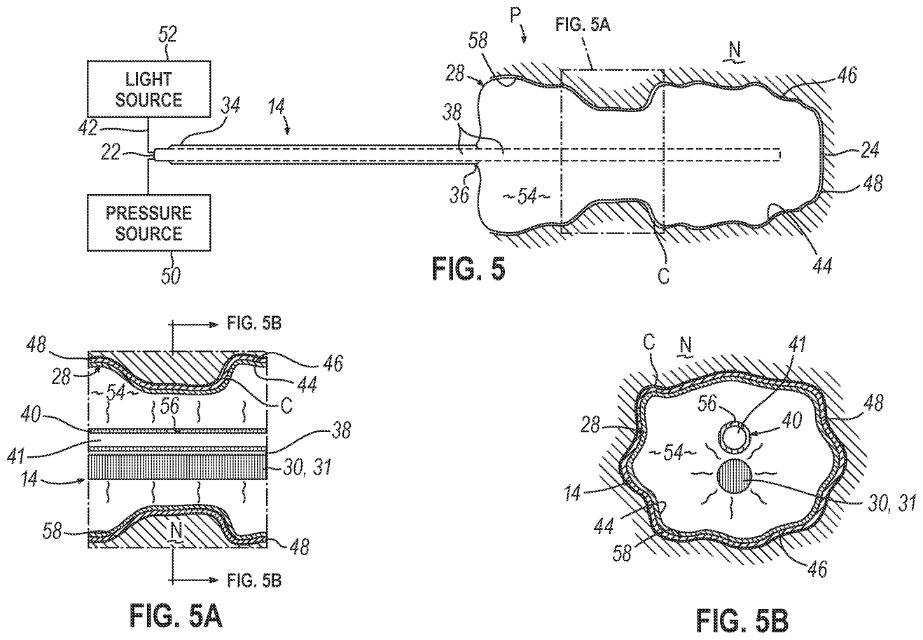

Balloon (28) is configured to extensively elongate when inflated from a contracted configuration to an expanded configuration without rupturing. For example, balloon (28) may be formed from a soft and highly stretchable polymer, such as silicone. The ability to extensively elongate without rupturing enables balloon (28) to closely conform to the shape of the target anatomical structure (C), which contrasts a typical sinuplasty balloon (not shown) that would not be generally suited for dilation if the sinuplasty balloon were extensible. Balloon (28) includes inner and outer surfaces (44, 46). As shown, outer surface (46) of balloon (28) may be at least partially coated with a photosensitizer (48). Alternatively, it is envisioned that the entire balloon (28) may be coated with photosensitizer (48). As will be described in greater detail with reference to FIGS. 5-5B, the target anatomical structure (C) may by ablated by activating photosensitizer (48) that at least partially coats outer surface (46) of balloon (28) using light emitting diode (LED) (31) to provide photodynamic therapy when balloon (28) is in the expanded configuration. As shown in FIGS. 2A-2B, 3A-3B, 4A-4B, 5A-5B, 6A-6B, and 7A-7B, photosensitizer (48) coats entire outer surface (46) of balloon (28). Photosensitizer (48) is not shown in FIG. 2, 3, 4, 5, 6, or 7.

A pressure source (50) and a light source (52) are configured to be operatively coupled with balloon catheter (14). As shown in FIG. 2, both pressure source (50) and light source (52) are positioned outside of the patient (P) and coupled with proximal portion (34) of shaft (26). Pressure source (50) may be coupled (e.g. in fluid communication) with balloon catheter (14) at any time prior to inflation, and pressure source (50) may be decoupled from balloon catheter (14) at any time after deflation. Pressure source (50), shown in FIG. 2, is removably coupled with lumen (38) or inflation tube (40) to inflate and/or deflate balloon (28) when desired.

Light source (52) may include an electrical power source that transmits electrical power through wire (42) that extends longitudinally to a light, shown as LED (31). While the light is shown as LED (31), a range of suitable light emitting light sources are also envisioned that suitably activate photosensitizer to perform photodynamic therapy. A single LED or multiple LEDs may be used in combination to achieve the desired light. As shown, LED (31) is disposed within an interior (54) of balloon (28). As such, light (e.g. LED (31)) may be configured to receive power from the light source (52) using wire (42) that extends through lumen (38) of shaft (26) as shown in FIG. 3. The light in balloon (28) may also be provided via the distal end of an optical fiber, an optical fiber bundle, a light pipe, or another light conveying structure, with light source (52) being optically coupled with a proximal portion of the optical fiber, optical fiber bundle, light pipe, etc.

As shown in FIGS. 3-3B, distal end (24) of balloon catheter (14) is distally advanced until balloon (28) is disposed in the target anatomical structure (C) of the ear (E), the nose (N), or the throat (T). Particularly, distal end (24) of balloon catheter (14) extends through the sinus ostium (O) and into the maxillary sinus (MS). FIG. 3 shows a schematic side elevational view of distal end (24) of balloon catheter (14) of FIG. 2, but with balloon (28) positioned in the target anatomical structure (C) (e.g. maxillary sinus (MS)) in the contracted configuration. FIG. 3A shows an enlarged sectional view of balloon catheter (14) within the maxillary sinus (MS) of FIG. 3. FIG. 3B shows a cross-sectional view of FIG. 3A, taken along line 3B-3B of FIG. 3A.

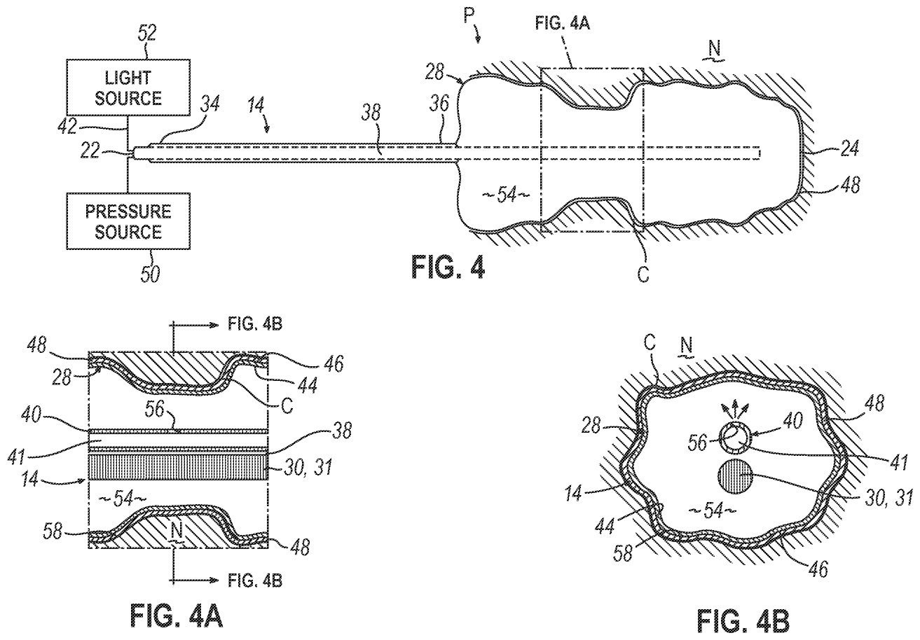

As shown in FIGS. 4-4B, the method includes inflating balloon to an expanded configuration in target anatomical structure (C) of the ear (E), the nose (N), or the throat (T). FIG. 4 shows a schematic side elevational view of distal end (24) of balloon catheter (14) positioned within the target anatomical structure (C) (e.g. maxillary sinus (MS)) of FIG. 3, but with a pressure source expanding balloon (28) to an expanded configuration. FIG. 4A shows an enlarged schematic sectional view of balloon catheter (14) within the target anatomical structure (C) of FIG. 4. FIG. 4B shows a cross-sectional view of FIG. 4A, taken along line 4B-4B of FIG. 4A.

With continued reference to FIGS. 4-4B, inflation fluid travels from pressure source (50), through lumen (41) defined by inflation tube (40) disposed within lumen (38), through port (56), and into interior (54) of balloon (28) as shown by the arrows. Alternatively, inflation fluid may travel from pressure source (50), through lumen (38) of shaft (26), and into interior (54) of balloon (28). Inflation fluid within interior (54) of balloon (28) causes balloon (28) to radially expand to the expanded configuration. Since balloon (28) is extensible, outer surface (46) of balloon (28) conforms to inner surface (58) of the target anatomical structure (C), such that the radial expansion may be non-uniform. Inflation fluid of pressure source (50) may be an incompressible liquid (e.g. saline, etc.) or a compressible gas. Balloon (28) may be inflated to a predetermined pressure. For example, the predetermined pressure may be selected so as to be great enough to sufficiently push balloon (28) into the curves of the target anatomical structure (C); but not too great so as to dilate the target anatomical structure (C).

In the expanded configuration, at least a portion of outer surface (46) of balloon (28) may be in contact with target anatomical structure (C). As previously described with reference to FIGS. 2-2B, photosensitizer (48) may cover a portion of outer surface (46) of balloon (28) or the entirety of outer surface (46) of balloon (28). As shown, when balloon (28) is in the expanded configuration, photosensitizer (48) is in direct contact with inner surface (58) of target anatomical structure (C) of the nose (N). Photosensitizer (48) may include hemoglobin; however, various other suitable photosensitizers, combinations of photosensitizers, or combinations of photosensitizers and non-photosensitizers are also envisioned. As such, outer surface (46) of balloon (28) may be at least partially coated with hemoglobin. Hemoglobin may be in direct contact with inner surface (58) of target anatomical structure (C) when balloon (28) is in the expanded configuration.

As shown in FIGS. 5-5B, the method includes ablating the target anatomical structure (C) using at least one of photodynamic therapy or heated inflation fluid when balloon (28) is in the expanded configuration. Photodynamic therapy may employ non-toxic dyes known as photosensitizers, which may absorb visible light to produce an excited singlet state, followed by a triplet state that may undergo photochemistry. In the presence of ambient oxygen, reactive oxygen species, such as singlet oxygen and hydroxyl radicals are formed that are capable of ablating tissue cells. In other words, when photosensitizer (48) receives light from LED (31) (or some other suitable light source), photosensitizer (48) releases oxygen that is capable of ablating tissue that is in contact with balloon (28). Once balloon (28) is in the expanded configuration in the target anatomical structure (C), the light may be activated. Photodynamic therapy (PDT) is suited for applications using a compliant balloon conforming to various geometries (e.g. target anatomical cavities having various shapes and sizes). As previously described, balloon (28) may be coated with a PDT agent (i.e. a photosensitizer). For example, photosensitizer (48) may include hemoglobin. The combination of the light (e.g. LED (31)) and the photosensitizer (48) ultimately causes the photodynamic therapy, thereby ablating the target anatomical structure (C) in the ear (E), the nose (N), or the throat (T) of the patient (P). In other words, exposing photosensitizer (48) to light (e.g. LED (31)) ultimately causes ablation of the target anatomical structure (C). Balloon (28) may be formed from a transparent or otherwise optically transmissive material, which enables light from LED (31) to penetrate through and activate photosensitizer (48).

Alternatively, inflation fluid within balloon (28) may be heated using heating element (32) (see FIG. 9) that may be provided through wire (64) from heat source (62) (see FIG. 9). Heat source (62) may be configured similar to light source (52) shown and described with reference to FIGS. 2-7B. The heated inflation fluid may be applied to target anatomical structure (C) of the ear (E), the nose (N), or the throat (T) of the patient (P). It is envisioned that heating element (32) may be disposed within balloon (28), such that heating element (32) may be in direct contact with non-conductive inflation fluid (not shown) that is disposed within interior of balloon (28). Heating element (32) may include a flexible resistance heating element or any other suitable kind of thermal heating element. Heating element (32) may be fixably coupled with balloon catheter (14). Heating element (32) may be disposed at a distal portion (36) of shaft (26) of balloon catheter (14).

In some versions where ablation feature (30) includes heating element (32), balloon (28) may be inflated with non-conductive inflation fluid (e.g. a D5W solution that includes 5% dextrose and 95% water) while balloon (28) is disposed within the target anatomical structure (C). The inflation fluid can be mixed via a flexible mixer attached at heating element (32) to ensure consistent ablation throughout the target anatomical cavity (e.g. sinus, ET, or any other cavity). The non-conductive inflation fluid may be heated to approximately 95 degrees Celsius using heating element (32). When using heated inflation fluid, balloon (28) may optionally be coated with hemoglobin. Balloon (28) may be formed from silicone, to the extent that such material may generally withstand temperatures above 100 degrees Celsius. Since cell ablation may begin above approximately 45 degrees Celsius, using heated inflation fluid at a temperature above that needed for cell ablation (yet below the melting point of the balloon (28)) may result in tissue ablation throughout the target anatomical structure (e.g. the sinus cavity, ET, or any other suitable structure of ENT).

FIG. 5 shows a schematic side elevational view of distal end (24) of balloon catheter (14) positioned within the target anatomical structure (C) of FIG. 4, but with ablation feature (30) (shown as LED (31)) providing light to ablate the target anatomical structure (C). FIG. 5A shows an enlarged schematic sectional view of balloon catheter (14) with ablation feature (30) (shown as LED (31)) disposed within balloon (28), when balloon (28) is disposed within the target anatomical structure (C) of FIG. 5. FIG. 5B shows a cross-sectional view of balloon catheter (14) with ablation feature (30) (shown as LED (31)) disposed within balloon (28), where balloon (28) is disposed within the target anatomical structure (C) of FIG. 5A taken along line 5B-5B of FIG. 5A. As previously described, it is also envisioned that heating element (32) of ablation feature (30) may be disposed within balloon (28), when balloon (28) is disposed within the target anatomical structure (C).

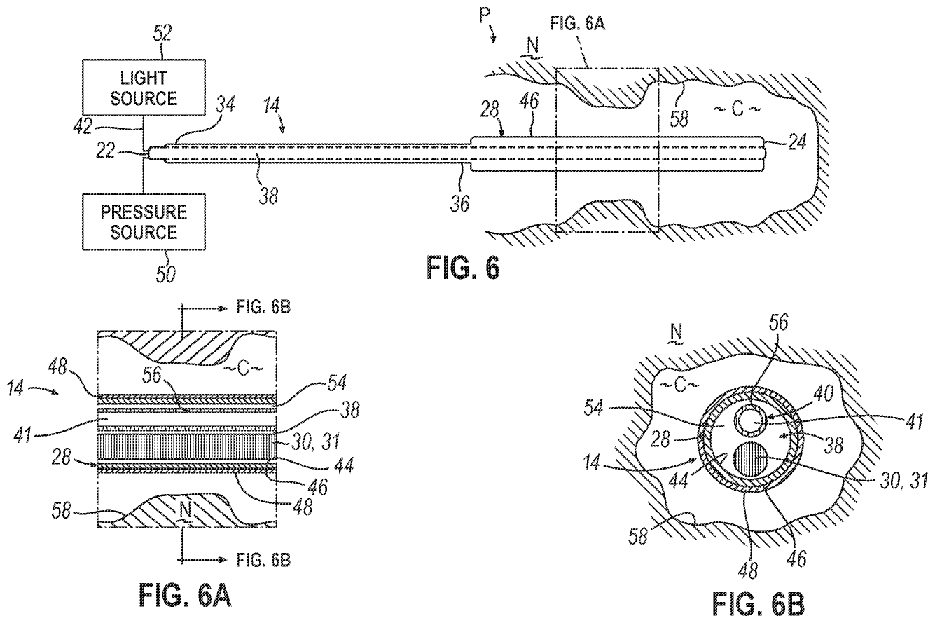

After the inner surface (58) of the targeted anatomical structure (C) has been sufficiently ablated, balloon (28) may be deflated to the contracted configuration. As shown in FIGS. 6-6B, the method may include removing balloon catheter (14) from patient (P) while balloon (28) is in the contracted configuration. FIG. 6 shows a schematic side elevational view of distal end (24) of balloon catheter (14) positioned within the target anatomical structure (C) of FIG. 5, but with pressure source (50) contracting balloon (28) to the contracted configuration. FIG. 6A shows an enlarged schematic sectional view of balloon catheter (14) within the target anatomical structure (C) of FIG. 6. FIG. 6B shows a cross-sectional view of balloon catheter (14) within the target anatomical structure (C) of FIG. 6A taken along line 6B-6B of FIG. 6A.

As shown in FIG. 6, balloon (28) is deflated causing balloon (28) to radially contract to the contracted configuration. This deflation may be achieved by fluidly coupling pressure source (50) (e.g. a vacuum) to lumen of shaft (26) and subsequently vacuuming inflation fluid out of interior of balloon (28). In some other variations, lumen is simply vented to atmosphere, relieving outward pressure of inflation fluid on interior (54) of balloon (28). In such variations, balloon (28) may partially collapse as balloon (28) is pulled out of the target anatomical structure (C), with balloon (28) at least temporarily conforming to the traversed walls of the target anatomical structure (C) during withdrawal of balloon (28) from the target anatomical structure (C). In some versions, balloon (28) is resiliently biased toward the contracted configuration, such that balloon (28) will resiliently return to the contracted configuration in response to pressure being relieved from interior (54) of balloon (28).

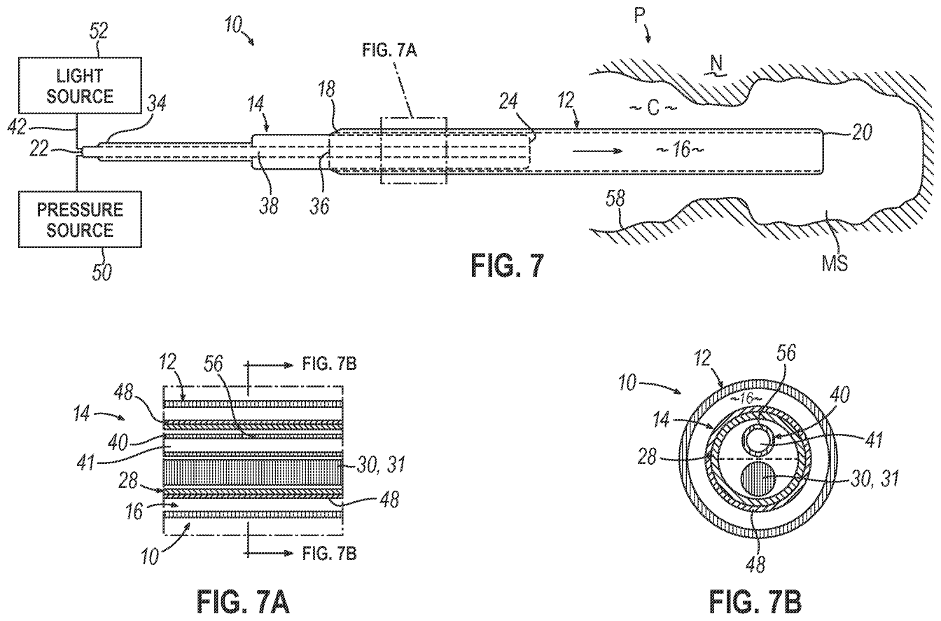

FIG. 7 shows a schematic side elevational view of distal end (24) of balloon catheter (14) being removed through lumen (16) of guide (12) of kit (10) from the target anatomical structure (C) of FIG. 6. In other words, distal end (24) of balloon catheter (14) is proximally withdrawn from the target anatomical structure (C) of the patient (P) while balloon (28) is in the contracted configuration. FIG. 7A shows an enlarged schematic sectional view of the target anatomical structure (C) of FIG. 7, and FIG. 7B shows a cross-sectional view of FIG. 7A taken along line 7B-7B of FIG. 7A. As shown in FIG. 7, balloon catheter (14) is removed from the patient (P) while balloon (28) is in the contracted configuration.

B. Exemplary Light Disposed at Distal Tip of Shaft

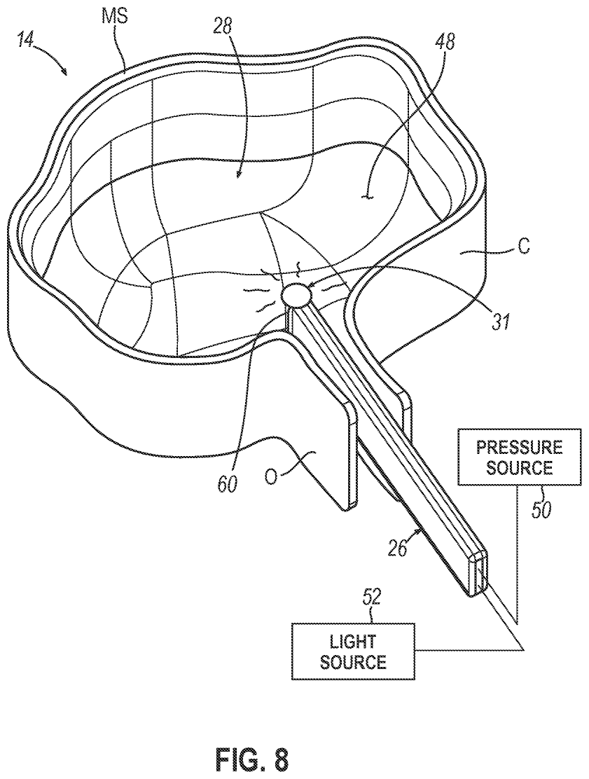

FIG. 8 shows a schematic perspective view of the balloon catheter (14) of FIG. 5 ablating the target anatomical structure (C), but with LED (31) positioned outside of balloon (28). As shown, LED (31) is positioned at a distal tip (60) of shaft (26). As shown, LED (31) is fixedly positioned in relation to balloon (28) which is shown in the expanded configuration. As shown, balloon catheter (14) is electrically coupled with light source (52). Additionally, the target anatomical structure (C) is shown as a sinus cavity; however, other target anatomical structures (C) are also envisioned.

As previously described with reference to FIGS. 5-5B, the target anatomical structure (C) may by ablated by activating photosensitizer (48) that at least partially coats outer surface (46) of balloon (28) using LED (31) to ultimately provide photodynamic therapy when the balloon (28) is in the expanded configuration. Balloon (28) may be formed from a transparent or otherwise optically transmissive material, which enables light from LED (31) to penetrate through and activate photosensitizer (48). Since balloon (28) is optically transmissive, light from LED (31) (or another suitable light source) may pass through the entirety of balloon (28), including the side of balloon (28) that is opposite to the LED (31). Moreover, the inflation fluid within balloon (28) may assist in conducting light from LED (31) to all surfaces of balloon (28).

C. Exemplary Heating Element Disposed at Distal Tip of Shaft

FIG. 9 shows a schematic perspective view of the balloon catheter (14) ablating the target anatomical structure (C) using heating element (32) that is positioned outside of balloon (28) which is shown in the expanded configuration. It is envisioned that heating element (32) may be disposed adjacent balloon (28), such that heating element (32) may be in indirect contact with non-conductive inflation fluid (not shown) that disposed within interior of balloon (28). The target anatomical structure (C) is shown as a sinus cavity. As shown, heating element (32) is positioned at distal tip (60) of shaft (26). As shown, balloon catheter (14) is coupled with a heat source (62) using a wire (64), instead of being coupled with light source (52) using wire (42) shown in FIG. 8. Inflation fluid within balloon (28) may be heated using heating element (32) that may be provided through wire (64) from heat source (62). Heating element (32) may include a flexible resistance heating element.

For example, balloon (28) may be inflated with non-conductive inflation fluid (e.g. a D5W solution that includes 5% dextrose and 95% water) while balloon (28) is disposed within the target anatomical structure (C). The inflation fluid can be mixed via a flexible mixer attached at heating element (32) to ensure consistent ablation throughout the target anatomical cavity (e.g. sinus, ET, or any other cavity). The non-conductive inflation fluid may be heated to approximately 95 degrees Celsius using heating element (32) as described above, resulting in tissue ablation throughout the target anatomical structure (e.g. the sinus cavity, ET, or any other suitable structure within the ear, nose, or throat).

D. Exemplary Method

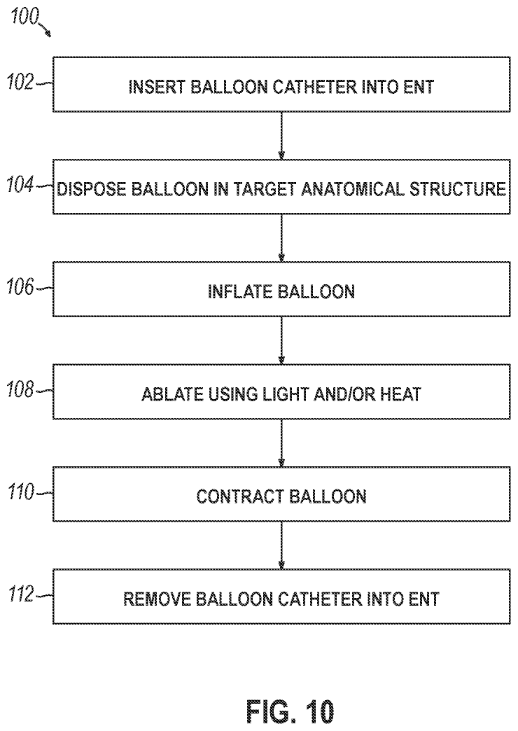

FIG. 10 shows an exemplary method (100) of ablating the target anatomical structure (C) in the ear (E), the nose (N), or the throat (T) of the patient (P). At step (102), method (100) includes inserting a distal end (24) of a balloon catheter (14) into the ear (E), the nose (N), or the throat (T) of the patient (P). This may be performed using a guide (e.g. guide (12)) or without using the guide.

At step (104), method (100) includes distally advancing distal end (24) of balloon catheter (14) until balloon (28) is disposed in the target anatomical structure (C) in the ear (E), the nose (N), or the throat (T) of the patient (P). At step (106), method (100) includes inflating balloon (28) to an expanded configuration in the target anatomical structure (C) of the ear (E), the nose (N), or the throat (T) of the patient (P). At this stage, the outer surface of balloon (28) is in full contact with all of the tissue that is intended to be ablated.

At step (108), method (100) includes ablating the target anatomical structure (C) in the ear (E), the nose (N), or the throat (T) using at least one of photodynamic therapy applied by LED (31) to the target anatomical structure (C) of the ear (E), the nose (N), or the throat (T); or inflation fluid within balloon (28) being heated by heating element (32), and that heat being transferred to the target anatomical structure (C) in the ear (E), the nose (N), or the throat (T). At step (110), method (100) includes deflating balloon (28) from the expanded configuration to the contracted configuration in the target anatomical structure (C) of the ear (E), the nose (N), or the throat (T). At step (112), method (100) includes proximally retracting distal end (24) of balloon catheter (14) from the target anatomical structure (C) in the ear (E), the nose (N), or the throat (T).

### II. Exemplary Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A method of ablating tissue of a target anatomical structure in an ear, a nose, or a throat of a patient, the method comprising: (a) inserting a distal end of a balloon catheter into the ear, the nose, or the throat of the patient, wherein the balloon catheter includes a balloon operatively coupled with a shaft; (b) distally advancing the distal end of the balloon catheter until the balloon is disposed in the target anatomical structure of the ear, nose, or throat of the patient; (c) inflating the balloon to an expanded configuration in the target anatomical structure of the ear, the nose, or the throat of the patient; and (d) ablating the tissue of the target anatomical structure of the ear, the nose, or the throat of the patient using at least one of: (i) photodynamic therapy where a photosensitizer is configured to ablate the tissue in response to exposure to light when the balloon is in the expanded configuration, or (ii) heated inflation fluid within the balloon, the inflation fluid being heated by a heating element when the balloon is in the expanded configuration.

### Example 2

The method of Example 1, wherein the target anatomical structure includes a sinus cavity or a Eustachian tube of the ear, nose, or throat of the patient.

### Example 3

The method of any one or more of Examples 1 through 2, wherein the balloon includes an outer surface at least partially coated with the photosensitizer, wherein ablating the target anatomical structure further comprises ablating the target anatomical structure of the ear, the nose, or the throat of the patient by activating the photosensitizer that at least partially coats the outer surface of the balloon using the light to provide the photodynamic therapy.

### Example 4

The method of any one or more of Examples 1 through 3, wherein the photosensitizer includes hemoglobin.

### Example 5

The method of any one or more of Examples 1 through 4, wherein the light is fixably coupled with the balloon catheter.

### Example 6

The method of any one or more of Examples 1 through 5, wherein the light is disposed along a distal portion of the shaft of the balloon catheter.

### Example 7

The method of any one or more of Examples 1 through 6, wherein the light includes at least one light emitting diode.

### Example 8

The method of any one or more of Examples 1 through 7, wherein the heating element includes a flexible resistance heating element.

### Example 9

The method of any one or more of Examples 1 through 8, wherein the heating element is fixably coupled with the balloon catheter.

### Example 10

The method of any one or more of Examples 1 through 9, wherein the heating element is disposed along a distal portion of the shaft of the balloon catheter.

### Example 11

The method of any one or more of Examples 1 through 2 and Examples 4 through 10, wherein inflating the balloon further comprises inflating the balloon to the expanded configuration such that at least a portion of an outer surface of the balloon is in contact with the target anatomical structure, wherein ablating the target anatomical structure of the ear, the nose, or the throat of the patient using the light or the heating element while the at least a portion of the outer surface of the outer balloon is in contact with the target anatomical structure of the ear, the nose, or the throat.

### Example 12

The method of any one or more of Examples 1 through 11, wherein inflating the balloon further comprises inflating the balloon to a predetermined pressure that does not dilate the target anatomical structure of the ear, the nose, or the throat.

### Example 13

The method of any one or more of Examples 1 through 12, wherein the balloon has an outer surface at least partially coated with hemoglobin, wherein inflating the balloon further comprises inflating the balloon such that the hemoglobin is in direct contact with an inner surface of the target anatomical structure of the ear, the nose, or the throat.

### Example 14

The method of any one or more of Examples 1 through 13, wherein the balloon is longitudinally and radially extensible and formed from a stretchable polymer.

### Example 15

The method of any one or more of Examples 1 through 14, further comprising: (a) inserting a distal end of a guide into the patient, wherein the guide includes a lumen; (b) distally advancing the distal end of the guide until the guide is near the target anatomical structure; and (c) subsequently inserting the distal end of the balloon catheter through the lumen of the guide into the patient.

### Example 16

A method of ablating tissue of a target sinus cavity in an ear, a nose, or a throat of a patient, the method comprising: (a) inserting a distal end of a balloon catheter into the ear, the nose, or the throat of the patient, wherein the balloon catheter includes an extensible balloon operatively coupled with a shaft; (b) distally advancing the distal end of the balloon catheter until the extensible balloon is disposed in the target sinus cavity of the ear, the nose, or the throat of the patient; (c) inflating the extensible balloon to an expanded configuration in the target sinus cavity of the ear, nose, or throat of the patient; and (d) ablating the tissue of the target sinus cavity of the ear, the nose, or the throat of the patient by: (i) exposing a photosensitizer on the extensible balloon to a light to cause photodynamic therapy that ablates the tissue, or (ii) activating a heating element to heat inflation fluid within the extensible balloon to thereby ablate the tissue.

### Example 17

The method of Example 16, wherein the extensible balloon includes an outer surface at least partially coated with the photosensitizer, wherein ablating the target sinus cavity further comprises ablating the target sinus cavity of the ear, the nose, or the throat of the patient by activating the photosensitizer that at least partially coats the outer surface of the extensible balloon in the expanded configuration using the light to provide the photodynamic therapy.

### Example 18

The method of any one or more of Examples 16 through 17, wherein the light or the heating element is disposed at the distal end of the balloon catheter.

### Example 19

The method of any one or more of Examples 16 through 18, wherein the light or the heating element is fixably coupled with the distal end of the balloon catheter.

### Example 20

A kit configured to ablate tissue of a target anatomical structure in an ear, a nose, or a throat of a patient comprising: (a) a guide sized and configured to fit near the target anatomical structure of the ear, the nose, or the throat of the patient, wherein the guide includes a lumen; and (b) a balloon catheter sized and configured to pass through the lumen of the guide and ablate the target anatomical structure in the ear, the nose, or the throat of the patient, wherein the balloon catheter comprises: (i) a shaft defining a longitudinal axis, wherein the shaft includes a proximal portion and a distal portion, (ii) an extensible balloon coupled with the distal portion of the shaft, wherein the extensible balloon is configured to extensively elongate when inflated from a contracted configuration to an expanded configuration, and (iii) at least one of a heating element or a light operatively coupled with the distal portion of the shaft and configured to ablate the target anatomical structure of the ear, the nose, or the throat of the patient using photodynamic therapy.

### III. Miscellaneous

It should be understood that any of the examples described herein may include various other features in addition to or in lieu of those described above. By way of example only, any of the examples described herein may also include one or more of the various features disclosed in any of the various references that are incorporated by reference herein.

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices disclosed herein can be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, versions of the device may be disassembled, and any number of the particular pieces or parts of the device may be selectively replaced or removed in any combination. Upon cleaning and/or replacement of particular parts, versions of the device may be reassembled for subsequent use either at a reconditioning facility, or by a surgical team immediately prior to a surgical procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be processed before surgery. First, a new or used instrument may be obtained and if necessary cleaned. The instrument may then be sterilized. In one sterilization technique, the instrument is placed in a closed and sealed container, such as a plastic or TYVEK bag. The container and instrument may then be placed in a field of radiation that can penetrate the container, such as gamma radiation, x-rays, or high-energy electrons. The radiation may kill bacteria on the instrument and in the container. The sterilized instrument may then be stored in the sterile container. The sealed container may keep the instrument sterile until it is opened in a surgical facility. A device may also be sterilized using any other technique known in the art, including but not limited to beta or gamma radiation, ethylene oxide, or steam.

Having shown and described various versions of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, versions, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. A method of ablating tissue of a target anatomical structure in an ear, a nose, or a throat of a patient, the method comprising:
(a) inserting a distal end of a balloon catheter into the ear, the nose, or the throat of the patient, wherein the balloon catheter includes a balloon operatively coupled with a shaft;
(b) distally advancing the distal end of the balloon catheter until the balloon is disposed in the target anatomical structure of the ear, nose, or throat of the patient;
(c) inflating the balloon to an expanded configuration in the target anatomical structure of the ear, the nose, or the throat of the patient; and
(d) ablating the tissue of the target anatomical structure of the ear, the nose, or the throat of the patient using at least one of:
(i) photodynamic therapy where a photosensitizer is configured to ablate the tissue in response to exposure to light when the balloon is in the expanded configuration, or
(ii) heated inflation fluid within the balloon, the inflation fluid being heated by a heating element when the balloon is in the expanded configuration.

2. The method of claim 1, wherein the target anatomical structure includes a sinus cavity or a Eustachian tube of the ear, nose, or throat of the patient.

3. The method of claim 1, wherein the balloon includes an outer surface at least partially coated with the photosensitizer, wherein ablating the target anatomical structure further comprises ablating the target anatomical structure of the ear, the nose, or the throat of the patient by activating the photosensitizer that at least partially coats the outer surface of the balloon using the light to provide the photodynamic therapy.

4. The method of claim 1, wherein the photosensitizer includes hemoglobin.

5. The method of claim 1, wherein the light is fixably coupled with the balloon catheter.

6. The method of claim 1, wherein the light is disposed along a distal portion of the shaft of the balloon catheter.

7. The method of claim 1, wherein the light includes at least one light emitting diode.

8. The method of claim 1, wherein the heating element includes a flexible resistance heating element.

9. The method of claim 1, wherein the heating element is fixably coupled with the balloon catheter.

10. The method of claim 1, wherein the heating element is disposed along a distal portion of the shaft of the balloon catheter.

11. The method of claim 1, wherein inflating the balloon further comprises inflating the balloon to the expanded configuration such that at least a portion of an outer surface of the balloon is in contact with the target anatomical structure, wherein ablating the target anatomical structure of the ear, the nose, or the throat of the patient using the light or the heating element while the at least a portion of the outer surface of the outer balloon is in contact with the target anatomical structure of the ear, the nose, or the throat.

12. The method of claim 1, wherein inflating the balloon further comprises inflating the balloon to a predetermined pressure that does not dilate the target anatomical structure of the ear, the nose, or the throat.

13. The method of claim 1, wherein the balloon has an outer surface at least partially coated with hemoglobin, wherein inflating the balloon further comprises inflating the balloon such that the hemoglobin is in direct contact with an inner surface of the target anatomical structure of the ear, the nose, or the throat.

14. The method of claim 1, wherein the balloon is longitudinally and radially extensible and formed from a stretchable polymer.

15. The method of claim 1, further comprising:
(a) inserting a distal end of a guide into the patient, wherein the guide includes a lumen;
(b) distally advancing the distal end of the guide until the guide is near the target anatomical structure; and
(c) subsequently inserting the distal end of the balloon catheter through the lumen of the guide into the patient.

16. A method of ablating tissue of a target sinus cavity in an ear, a nose, or a throat of a patient, the method comprising:
(a) inserting a distal end of a balloon catheter into the ear, the nose, or the throat of the patient, wherein the balloon catheter includes an extensible balloon operatively coupled with a shaft;
(b) distally advancing the distal end of the balloon catheter until the extensible balloon is disposed in the target sinus cavity of the ear, the nose, or the throat of the patient;
(c) inflating the extensible balloon to an expanded configuration in the target sinus cavity of the ear, nose, or throat of the patient; and
(d) ablating the tissue of the target sinus cavity of the ear, the nose, or the throat of the patient by:
(i) exposing a photosensitizer on the extensible balloon to a light to cause photodynamic therapy that ablates the tissue, or
(ii) activating a heating element to heat inflation fluid within the extensible balloon to thereby ablate the tissue.

17. The method of claim 16, wherein the extensible balloon includes an outer surface at least partially coated with the photosensitizer, wherein ablating the tissue of the target sinus cavity further comprises ablating the tissue by activating the photosensitizer that at least partially coats the outer surface of the extensible balloon in the expanded configuration.

18. The method of claim 16, wherein the light or the heating element is disposed at the distal end of the balloon catheter.

19. The method of claim 16, wherein the light or the heating element is fixably coupled with the distal end of the balloon catheter.

20. A kit configured to ablate tissue of a target anatomical structure in an ear, a nose, or a throat of a patient comprising:
(a) a guide sized and configured to fit near the target anatomical structure of the ear, the nose, or the throat of the patient, wherein the guide includes a lumen; and
(b) a balloon catheter sized and configured to pass through the lumen of the guide and ablate the target anatomical structure in the ear, the nose, or the throat of the patient, wherein the balloon catheter comprises:
(i) a shaft defining a longitudinal axis, wherein the shaft includes a proximal portion and a distal portion,
(ii) an extensible balloon coupled with the distal portion of the shaft, wherein the extensible balloon is configured to extensively elongate when inflated from a contracted configuration to an expanded configuration, and
(iii) at least one of a heating element or a light operatively coupled with the distal portion of the shaft and configured to ablate the target anatomical structure of the ear, the nose, or the throat of the patient using photodynamic therapy or heated inflation fluid within the extensible balloon.
